(19) United States
(12) Patent Application Publication
(10) Pub. No.: US 2022/0033354 A1
(43) Pub. Date: Feb. 3, 2022

(54) CATIONS AS CATALYST IN THE PRODUCTION OF ALKANE SULFONIC ACIDS

(71) Applicant: BASF SE, Ludwigshafen am Rhein (DE)

(72) Inventors: Timo OTT, Duisburg (DE); Ingo BIERTUEMPEL, Duisburg (DE)

(21) Appl. No.: 17/279,450

(22) PCT Filed: Sep. 19, 2019
(86) PCT No.: PCT/EP2019/075233

§ 371 Date: Mar. 24, 2021

(30) Foreign Application Priority Data
Sep. 25, 2018 (EP) 18196520.3

(51) Int. Cl. C07C 303/06; C07C 303/44

(52) U.S. Cl. CPC C07C 303/06; C07C 303/44

(57) ABSTRACT

The present invention relates to the use of cations or compounds forming stable cations in the production of alkane sulfonic acids as well as methods to produce methane sulfonic acids employing the cations as catalyst.

The present invention relates to the use of cations or compounds forming stable cations in the production of alkane sulfonic acids as well as methods to produce methane sulfonic acids employing the cations as catalyst.

Alkane sulfonic acids are organic acids that can reach a similar acid strength as that of inorganic mineral acids, for example, sulfuric acid. However, in contrast to usual mineral acids such as sulfuric and nitric acids, the sulfonic acids are non-oxidizing and do not give off vapors that are harmful to health, as can be observed with hydrochloric and nitric acids. Further, many sulfonic acids, for example, methane sulfonic acid, are biologically degradable. The applications of sulfonic acids are many, for example, in cleaning agents, surfactants, galvanic and electronic industry, as catalysts, and in organic synthesis, pharmaceutical chemistry, for example, as protective groups. The salts of sulfonic acids are employed, for example, as surfactants, for example, sodium dodecylsulfonate, or in the electroplating industry, especially as tin, zinc, silver, lead and indium, but also other metal, alkylsulfonates. Furthermore, organic salts are employed in pharmaceutical chemistry. The very high solubility of alkyl sulfonates plays an important role, in particular. Further, no harmful gases are formed in electrolysis, and the use of toxic compounds, for example, cyanide, which is common in many cases, is dispensed with.

The structurally simplest representative of alkane sulfonic acids is methane sulfonic acid. U.S. Pat. No. 2,493,038 describes the preparation of methane sulfonic acid from SO₃ and methane. US 2005/0070614 describes further methods for preparing methane sulfonic acid, and its application. The methods known in the prior art are in part complicated, cost-intensive, and lead to undesirable products because of the harsh reaction conditions.

The reaction conditions in conventional processes of alkanesulfonic acid production can result in undesirable side products, which even manifest themselves as disturbing inhibitors in the production of alkanesulfonic acids. This may lead to termination of the actual reaction for preparing the alkanesulfonic acid, but also to impurities, formation of side products and poor yields, based on sulfur trioxide and methane.

WO 2007/136425 A2 discloses the use of the compound di(methanesulfonyl) peroxide (DMSP), which must be prepared by a complex electrolysis and, in addition, is a crystallizable highly explosive solid, as an initiator in a reaction in which methanesulfonic acid is formed from sulfur trioxide and methane.

WO 2015/071365 A1 and WO 2015/071455 A1 both describe processes for the sulfonation of alkanes. The main steps are:

### SYNTHESIS OF AN INITIATOR/INITIATOR-SOLUTION

Preparation of a sulfur trioxide-solution (oleum) by dissolving sulfur trioxide in an inert solvent (e.g. sulfuric acid)

Reaction of oleum with the corresponding alkane after or during addition of the initiator/initiator-solution in a high-pressure-reactor.

Quenching of non-reacted starting material

Purification (e.g. distillation, crystallization etc.)

Recycling of the inert solvent (e.g. sulfuric acid).

According to said prior art, the initiator is particularly prepared by reacting an alkane sulfonic acid R—SO₃H, i.e. the desired product, with hydrogen peroxide to form an initiator-precursor R—SO₂—O—OH. Said initiator-precursor is then reacted with SO₃ yielding initiator compounds such as R—SO₂—O—O—SO₃H. The cited prior art therefore requires some amount of the desired product to form an initiator.

It is thus the object of the present invention to provide novel catalysts for the homogeneous catalysis in the preparation of alkane sulfonic acids, especially methane sulfonic acid (MSA). Particularly, it is the object of the invention to provide catalysts that do not require the desired product itself to be present as an initiator-precursor. Further, requirements for sulfurtrioxide and alkanes should be of no relevance, meaning that not only absolute pure raw materials might be used, but that impurities do not affect negatively the reaction.

In a first embodiment, the object of the present invention is solved by the use of a cation being stable under acid or super acid conditions as catalyst in the preparation of alkane sulfonic acids from alkanes and sulfur trioxide, especially in the preparation of methane sulfonic acid from methane and sulfur trioxide, said cation being able to react with the alkane to form an alkyl cation. The alkyl cation will afterwards react with sulfur trioxide forming the alkane sulfonic acid. Particularly, methane, ethane, propane, butane, isopropane, isobutane or a higher alkane can be reacted with sulfur trioxide to form the corresponding alkane sulfonic acid. Higher alkane within the meaning of the present application are straight or branched alkanes with 20 C-atoms or less.

Surprisingly it has been found that cations which are stable under acid or super acid conditions are able to react under said conditions with the alkane ALK and form an alkane cation:

Y⁺+ALK→HY+ALK⁺ (R1)

The cation Y⁺ may be either a stable cation and added as cation to the reaction solution. Alternatively, the cation can form in situ during the reaction according to the following reaction scheme:

H⁺+A→HX+Y⁺ (R2)

The cation is used in the production of alkane sulfonic acid from an alkane and sulfur trioxide. The reaction conditions are strongly acid or even super acid in his reaction. A super acid is an acid with an acidity greater than that of 100% pure sulfuric acid. Strongly acid means an acidity of 100% pure sulfuric acid or at least similar to it.

If the cation is stable, stability in this context means that it is able to react with the alkane but does not decompose within 24 h at room temperature (20° C.), i.e. the half-life time t_{1/2} at room temperature is at least 24 h, preferably at least 30 h, especially at least 48 h.

Stable cations are formed prior to their use, i.e. prior to their addition into the reactor in which the reaction between alkane and sulfur trioxide takes place. Preferably, one type of cation is used alone and not together with another type of cation.

Alternatively, the cation is produced in situ during the production of the alkane sulfonic acid. In such cases a compound is added to the reaction and the cation is formed according to the above shown reaction (R2). Suitable compounds to be used are halogens, especially I₂ and Br₂, inter halogen compounds, especially I-Br, or solid elements of the 15th or 16th group of the periodic table of elements, especially S, Se, Te, P, As, Sb.

If halogens or interhalogens are used as compounds, the bond between the halogens breaks heterolytically. Iodine would thus react to HI and I⁺, bromine to HBr and Br⁺, and the interhalogen either to HI and Br⁺ or to HBr and I⁺. The reactive cation would be I⁺ or Br⁺, which is formed in situ, and afterwards reacts with the alkane to the alkane cation as schematically shown above in (R1).

If solid elements of the 15^{th} or 16^{th} group of the periodic table are used, they may form oligomeric or polymeric cationic compounds, i.e., sulfur will form an oligomer Sₙ-S⁺, wherein n may for example be in the range of from 0 to 10, preferably from 2 to 10, or in another range. Said Sₙ-S⁺ would be the reactive compound. Similar compounds may also occur with the other elements. Alternatively, they can form cations without polymerisation/oligomerisation. To sum up all possible cations of S, Se, Te, As, Sb and P, they are summarized with S⁺, Se⁺, Te⁺, As⁺, Sb⁺, and P⁺ respectively. Additionally, also Silicon may be added to the reaction solution forming Si+ as cation.

In another embodiment, the object of the present invention is solved by a process for the preparation of alkane sulfonic acids, especially of methane sulfonic acid, comprising the steps of

i) providing sulfur trioxide and an alkane, especially methane, to a reaction chamber,

ii) setting a pressure of from 1 to 200 bar to the reactor,

iii) introducing a compound forming in situ a cation into said reactor,

iv) controlling the temperature in the reactor to be at 0° C. to 100° C.,

v) after the reaction is finished, if necessary, purifying the reaction product.

In a further embodiment, the object of the present invention is solved by a process for the preparation of alkane sulfonic acids, especially of methane sulfonic acid, comprising the steps of

i) providing sulfur trioxide and an alkane, especially methane, to a reaction chamber,

ii) setting a pressure of from 1 to 200 bar to the reactor,

iii) introducing a cation being stable under super acid conditions into said reactor,

iv) controlling the temperature in the reactor to be at 0° C. to 100° C.,

v) after the reaction is finished, if necessary, purifying the reaction product.

The reaction product in both cases is a mixture of alkane sulfonic acid, sulfuric acid and potentially an excess of SO₃. Further, impurities of the catalyst may be used. To obtain the pure alkane sulfonic acid, especially methane sulfonic acid, the reaction product is purified, especially by distillation.

Sulfur trioxide may be provided in the form of oleum, i.e., a solution of sulfur trioxide in sulfuric acid. Instead of oleum also pure sulfur trioxide can be employed. This avoids the preparation of sulfur trioxide solutions. The reaction conditions are here without added solvents. Further, non-reacted sulfur trioxide can evaporate, avoiding the necessity of quenching it.

In a further embodiment, sulfur trioxide is used in a form of oleum with a trioxide content of 50% (w/w) or less, or 65% (w/w) or more. Surprisingly it has been found that for the processes of the present invention also oleum with a sulfur trioxide content of 65% (w/w) or more, especially of 70% w/w or more can be used without negatively affecting the inventive process. Even pure sulfur trioxide (100% (w/w) sulfur trioxide) may be used.

The temperature during the reaction is preferably within a range from above 0° C. to 70° C., especially from 10° C. to 65° C., preferably from 20° C. to 60° C. Surprisingly the formation of side products was lower at lower temperatures. If the temperature is around 0° C. or 10° C., the reaction takes place but needs a longer time so that for an economically process the temperature is preferably 20° C. or above, especially about 40° C. to 55° C.

The pressure is set to be within a range from 1 to 200 bar, preferably from 50 to 150 bar, especially from 80 to 120 bar.

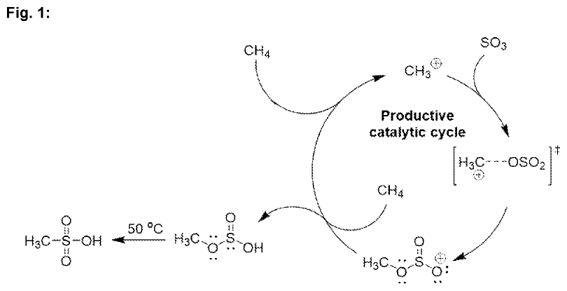

Without being bound to theory, in the process of the invention, the cation formed in situ or added in step iii) reacts with the alkane to form an alkane cation, said alkane cation reaction with SO₃ to form an alkane sulfur cation, said alkane sulfur cation reacting again with the alkane to form the alkane sulfonic acid and again an alkane cation. Therefore, the process according to the invention has in a first step the formation of a alkane cation ALK⁺. In case methane is used as alkane, a CH₃⁺ cation is formed which then reacts according to the reaction scheme depicted in FIG. 1.

The alkane in the process according to the invention can be any straight chain or branched alkane, preferably with 20 C-atoms or less. Especially preferred, the alkane is selected from methane, ethane, propane, butane, isopropane or isobutane. Accordingly, the respective alkyl sulfonic acid is formed. Preferably, the alkane is methane and the alkyl sulfonic acid is methane sulfonic acid. In this embodiment, the alkane cation is CH₃⁺, as shown in FIG. 1.

In another embodiment, the object of the present invention is solved by a mixture comprising an alkane, sulfur trioxide, a cation being able to react with the alkane to form an alkane cation, and optionally a solvent. In an alternative embodiment, the object of the present invention is solved by a mixture comprising an alkane, sulfur trioxide, a compound forming in situ a cation under super acid conditions, said cation being able to react with the alkane to form an alkane cation, and optionally a solvent.

Preferably, the solvent is sulfuric acid and/or the alkane it methane.

1. A process for preparation of alkane sulfonic acids from alkanes and sulfur trioxide, comprising reacting a cation being stable under super acid conditions with alkane to form an alkyl cation, wherein stability means that the cation is able to react with the alkane but does not decompose within 24 h at room temperature (20° C.), and wherein the cation is a halogen cation.

2. The process according to claim 1, wherein the cation is formed in situ in acid or super acid conditions during the preparation of alkane sulfonic acids.

3. The process according to claim 1, wherein the cation is formed prior to addition to the reaction for obtaining an alkane sulfonic acid.

4. The process according to claim 1, wherein the halogen cation is I+ and Br+, S+, Se+, Te+, As+, Sb+, P+ and/or Si+.

5. Process for the preparation of alkane sulfonic acids, comprising the steps of
i) providing sulfur trioxide and an alkane to a reaction chamber,
ii) setting a pressure of from 1 to 200 bar to the reactor,
iii) introducing a compound forming in situ a cation into said reactor, wherein the cation is a halogen cation,
iv) controlling the temperature in the reactor to be at 0° C. to 100° C., and
v) after the reaction is finished, if necessary, purifying the reaction product.

6. Process for the preparation of alkane sulfonic acids, comprising the steps of
i) providing sulfur trioxide and an alkane to a reaction chamber,
ii) setting a pressure of from 1 to 200 bar to the reactor,
iii) introducing a cation being stable under super acid conditions into said reactor, wherein stability means that the cation is able to react with the alkane but does not decompose within 24 h at room temperature (20° C.), and wherein the cation is a halogen cation,
iv) controlling the temperature in the reactor to be at 0° C. to 100° C., and
v) after the reaction is finished, if necessary, purifying the reaction product.

7. Process according to claim 5, wherein the purification is performed by distillation.

8. Process according to claim 5, wherein the sulfur trioxide is provided as oleum with a sulfur trioxide content of above 0% by weight to 65% by weight or as pure sulfur trioxide.

9. Process according to claim 5, wherein the temperature is within a range of from above 0° C. to 70° C.

10. Process according to claim 5, wherein the
cation formed in situ or added in step iii) reacts with the alkane to form an alkane cation, said alkane cation reaction with SO₃ to form an alkane sulfur cation,
said alkane sulfur cation reacting again with the alkane to form the alkane sulfonic acid and again an alkane cation.

11. Process according to claim 10, wherein the alkane is methane, the alkane sulfonic acid is methane sulfonic acid, and the alkane cation is CH₃⁺.

12. A mixture comprising an alkane, sulfur trioxide, a cation being able to react with the alkane to form an alkane cation, wherein the cation is a halogen cation, and optionally a solvent.

13. A mixture comprising an alkane, sulfur trioxide, a compound forming in situ a cation under super acid conditions, said cation being able to react with the alkane to form an alkane cation, wherein the cation is a halogen cation, and optionally a solvent.

14. The mixture according to claim 12, wherein the solvent is sulfuric acid and/or the alkane it methane.

15. The process of according to claim 5, wherein the process is for the preparation of methane sulfonic acid and step i) comprises providing sulfur trioxide and methane to a reaction chamber.

16. The process according to claim 5, wherein the halogen cation is I+ and Br+, S+, Se+, Te+, As+, Sb+, P+ and/or Si+.

17. The process of according to claim 6, wherein the process is for the preparation of methane sulfonic acid and step i) comprises providing sulfur trioxide and methane to a reaction chamber.

18. The process according to claim 6, wherein the halogen cation is I+ and Br+, S+, Se+, Te+, As+, Sb+, P+ and/or Si+.

19. The mixture according to claim 12, wherein the halogen cation is I+ and Br+, S+, Se+, Te+, As+, Sb+, P+ and/or Si+.

20. The mixture according to claim 13, wherein the halogen cation is I+ and Br+, S+, Se+, Te+, As+, Sb+, P+ and/or Si+.
